(19) United States
(12) Patent Application Publication
(10) Pub. No.: US 2005/0015004 A1
(43) Pub. Date: Jan. 20, 2005

(54) Systems and methods for combining an anatomic structure and metabolic activity for an object

(76) Inventors: Sarah Rose Hertel, Pewaukee, WI (US); Mark Kenneth Limkeman, Brookfield, WI (US); Robert James Young, San Francisco, CA (US); Robert Dana Milnes, Brookfield, WI (US)

Correspondence Address: Dean D. Small;Armstrong Teasdale LLP, Suite 2600, One Metropolitan Square, St. Louis, MO, 63102, US

(21) Appl. No.: 10/621,667

(22) Filed: Jul. 17, 2003

(51) Int. Cl. A61B 5/05

(52) U.S. Cl. 600/425; 600/427; 600/436

(57) ABSTRACT

A method for combining an anatomic structure and metabolic activity for an object is described. The method includes acquiring a first set of images by scanning the object using a first modality, acquiring a second set of images by scanning the object using a second modality, fusing the first and second sets of images to form a fused volume, identifying a region of interest (ROI) in the fused volume, the ROI corresponding to an organ of interest of the object, and providing a viewing path through the fused volume at least partially following the ROI.

### BACKGROUND OF THE INVENTION

This invention relates generally to imaging systems and more particularly to systems and methods for combining an anatomic structure and metabolic activity for an object.

Many deaths due to cancer are attributable to colorectal cancer (CRC). Prevalence of CRC in people over fifty years in age increases. Incomplete prevalence not leading to maturity of CRC increases with age. However, cancer occurrence decreases after polepectomy, which is a removal of polyps. It is believed that many cancers arise from pre-existing adenomatous polyps. Detection and removal of these polyps can prevent CRC from occurring, and has been associated with a reduction in the prevalence of CRC, and CRC mortality.

Widespread colorectal screening and preventive efforts are hampered by several practical impediments. For example, fecal occult blood testing and sigmoidoscopy have been shown to be insensitive in 50% or more of patients. This insensitivity is because lesions either do not bleed or bleed sporadically, and half of all polyps are above the reach of a sigmoidoscope. The results of barium enema examinations are dependent on proper technique, and considerable experience is required to gain accurate results.

Colonoscopy, considered by some physicians to be a standard of reference for colon screening, can have serious complications and is expensive. Moreover, a colonoscopy is an inconvenient and uncomfortable procedure for the patient. For example, one or two days before the colonoscopy, the patient is usually required to stop eating solid foods and drink only clear liquids, such as water. The patient may be required to take a laxative the day before the colonoscopy, and may be required to take a laxative on the day of the colonoscopy. During the procedure, a camera is inserted into a colon of the patient to afford a visual inspection of the interior of the colon. The patient is sedated which may also cause discomfort and nausea post-examination.

### BRIEF DESCRIPTION OF THE INVENTION

In one aspect, a method for combining an anatomic structure and metabolic activity for an object is described. The method includes acquiring a first set of images by scanning the object using a first modality, acquiring a second set of images by scanning the object using a second modality, fusing the first and second sets of images to form a fused volume, identifying a region of interest (ROI) in the fused volume, the ROI corresponding to an organ of interest of the object, and providing a viewing path through the fused volume at least partially following the ROI.

In another aspect, a computer-readable medium encoded with a program is described. The program is configured to instruct a computer to fuse at least two of computed tomography (CT) data, single photon emission computed tomography (SPECT) data, and positron emitted tomography (PET) images to form a fused data set, identify an ROI in the fused data set, the ROT corresponding to an organ of interest of an object, and provide a path through the fused data set along which to view the fused data set.

In yet another aspect, a computer is described. The computer is programmed to fuse CT images and PET images to form a fused volume, identify an ROI in the fused volume, the ROI corresponding to an organ of interest of the object, and provide a viewing path through the fused volume at least partially following the ROI.

In still another aspect, an imaging system for combining an anatomic structure and metabolic activity for an object is described. The imaging system includes a radiation source, a radiation detector, and a controller operationally coupled to the radiation source and the radiation detector. The controller is configured to acquire CT images generated by performing a CT colonography, acquire PET images generated by performing a PET scan of a colon of the object, fuse the CT images and PET images to form a fused volume, identify an ROI in the fused volume, the ROI corresponding to the colon, and provide a viewing path through the fused volume of interest partially following the ROI.

In another aspect, an imaging system for combining an anatomic structure and metabolic activity for an object is described. The imaging system includes a radiation source, a radiation detector, and a controller operationally coupled to the radiation source and the radiation detector. The controller is configured to acquire CT images generated by scanning the object using a first modality, acquire PET images generated by scanning the object using a second modality, fuse the CT images and PET images to form a fused volume, identify an ROI in the fused volume, the ROI corresponding to an organ of interest of the object, and provide a viewing path through the fused volume at least partially following the ROI.

### BRIEF DESCRIPTION OF THE DRAWINGS

FIG. 1 is a pictorial view of a computed tomography (CT) imaging system in which methods for combining an anatomic structure and metabolic activity for an object are implemented.

FIG. 2 is a block schematic diagram of the CT imaging system illustrated in FIG. 1.

FIG. 3 is an isometric view of an embodiment of a PET imaging system in which methods for combining an anatomic structure and metabolic activity for an object are implemented.

FIG. 4 is a block diagram of the PET imaging system of FIG. 3.

FIG. 5 is a flowchart of an embodiment of a method for combining an anatomic structure and metabolic activity for an object.

FIG. 6 shows an image of a colon of a patient to illustrate the method of FIG. 5.

### DETAILED DESCRIPTION OF THE INVENTION

In computed tomography (CT) imaging system configurations, an X-ray source projects a fan-shaped beam which is collimated to lie within an X-Y plane of a Cartesian coordinate system and generally referred to as an “imaging plane”. The X-ray beam passes through an object being imaged, such as a patient. The beam, after being attenuated by the object, impinges upon an array of radiation detectors. The intensity of the attenuated radiation beam received at the detector array is dependent upon the attenuation of an X-ray beam by the object. Each detector element of the array produces a separate electrical signal that is a measurement of the beam intensity at the detector location. The intensity measurements from all of the detectors are acquired separately to produce a transmission profile.

In third generation CT systems, the X-ray source and the detector array are rotated with a gantry within the imaging plane and around the object to be imaged such that the angle at which the X-ray beam intersects the object constantly changes. A group of X-ray attenuation measurements, i.e., projection data, from the detector array at one gantry angle is referred to as a “view”. A “scan” of the object comprises a set of views made at different gantry angles, or view angles, during one revolution of the X-ray source and detector.

In an axial scan, the projection data is processed to construct an image that corresponds to a two dimensional slice taken through the object. One method for reconstructing an image from a set of projection data is referred to in the art as the filtered back projection technique. This process converts the attenuation measurements from a scan into integers called “CT numbers” or “Hounsfield units”, which are used to control the brightness of a corresponding pixel on a cathode ray tube display. Positron emission tomography (PET) scanners incorporate a process similar to that found in CT, in that a map or the object attenuation can be generated. A method to perform this attenuation measurement includes use of rotation rod sources containing positron-emitting radionuclides. The rods rotate outside the patient bore, but inside the diameter of the PET detector ring. Annihilation events occurring in the rods can send one photon into a near-side detector while the pair photon traverses the object of interest in a manner similar to the CT X-ray. The data found from this method contains essentially the same information as that found from the CT method except for the statistical quality of the resultant data. In the rotating rod case, the statistical quality is orders of magnitude inferior to most common CT scans. For the PET purpose, data acquired in this manner is used to correct for the attenuation seen in the object by the 511 keV photons, which is often the most substantial correction performed on the PET data.

To reduce the total scan time, a “helical” scan may be performed. To perform a “helical” scan, the patient is moved while the data for the prescribed number of slices is acquired. Such a system generates a single helix from a fan beam helical scan. The helix mapped out by the fan beam yields projection data from which images in each prescribed slice may be reconstructed.

Reconstruction algorithms for helical scanning typically use helical weighing algorithms that weight the collected data as a function of view angle and detector channel index. Specifically, prior to a filtered backprojection process, the data is weighted according to a helical weighing factor, which is a function of both the gantry angle and detector angle. The weighted data is then processed to generate CT numbers and to construct an image that corresponds to a two dimensional slice taken through the object.

At least some CT systems are configured to also perform Positron Emission Tomography (PET) and are referred to as PET-CT systems. Positrons are positively charged electrons (anti-electrons) which are emitted by radio nuclides that have been prepared using a cyclotron or other device. The radionuclides most often employed in diagnostic imaging are fluorine-18 (¹⁸F), carbon-11 (¹¹C), nitrogen-13 (¹¹N), and oxygen-15 (¹⁵O). Radionuclides are employed as radioactive tracers called “radiophannaceuticals” by incorporating them into substances such as glucose or carbon dioxide. One common use for radiopharmaceuticals is in the medical imaging field.

To use a radiopharmaceutical in imaging, the radiopharmaceutical is injected into a patient and accumulates in an organ, vessel or the like, which is to be imaged. It is known that specific radiopharmaceuticals become concentrated within certain organs or, in the case of a vessel, that specific radiopharmaceuticals will not be absorbed by a vessel wall. The process of concentrating often involves processes such as glucose metabolism, fatty acid metabolism and protein synthesis. Hereinafter, in the interest of simplifying this explanation, an organ to be imaged including a vessel will be referred to generally as an “organ of interest” and the invention will be described with respect to a hypothetical organ of interest.

After the radiopharmaceutical becomes concentrated within an organ of interest and while the radionuclides decay, the radionuclides emit positrons. The positrons travel a very short distance before they encounter an electron and, when the positron encounters an electron, the positron is annihilated and converted into two photons, or gamma rays. This annihilation event is characterized by two features which are pertinent to medical imaging and particularly to, medical imaging using PET. First, each gamma ray has an energy of approximately 511 keV upon annihilation. Second, the two gamma rays are directed in nearly opposite directions.

In PET imaging, if the general locations of annihilations can be identified in three dimensions, a three dimensional image of radiopharmaceutical concentration in an organ of interest can be reconstructed for observation. To detect annihilation locations, a PET camera is employed. An exemplary PET camera includes a plurality of detectors and a processor which, among other things, includes coincidence detection circuitry.

The coincidence circuitry identifies essentially simultaneous pulse pairs which correspond to detectors which are essentially on opposite sides of the imaging area. Thus, a simultaneous pulse pair indicates that an annihilation has occurred on a straight line between an associated pair of detectors. Over an acquisition period of a few minutes millions of annihilations are recorded, each annihilation associated with a unique detector pair. After an acquisition period, recorded annihilation data can be used via any of several different well known image reconstruction methods to reconstruct the three dimensional image of the organ of interest.

As used herein, an element or step recited in the singular and preceded with the word “a” or “an” should be understood as not excluding plural the elements or steps, unless such exclusion is explicitly recited. Furthermore, references to “one embodiment” of the present invention are not intended to be interpreted as excluding the existence of additional embodiments that also incorporate the recited features.

Also as used herein, the phrase “reconstructing an image” is not intended to exclude embodiments of the present invention in which data representing an image is generated but a viewable image is not. Therefore, as used herein the term “image” broadly refers to both viewable images and data representing a viewable image. However, many embodiments generate (or are configured to generate) at least one viewable image.

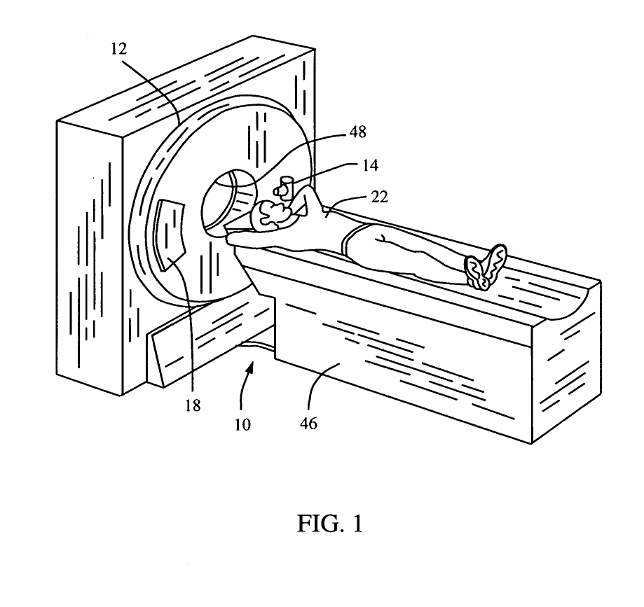

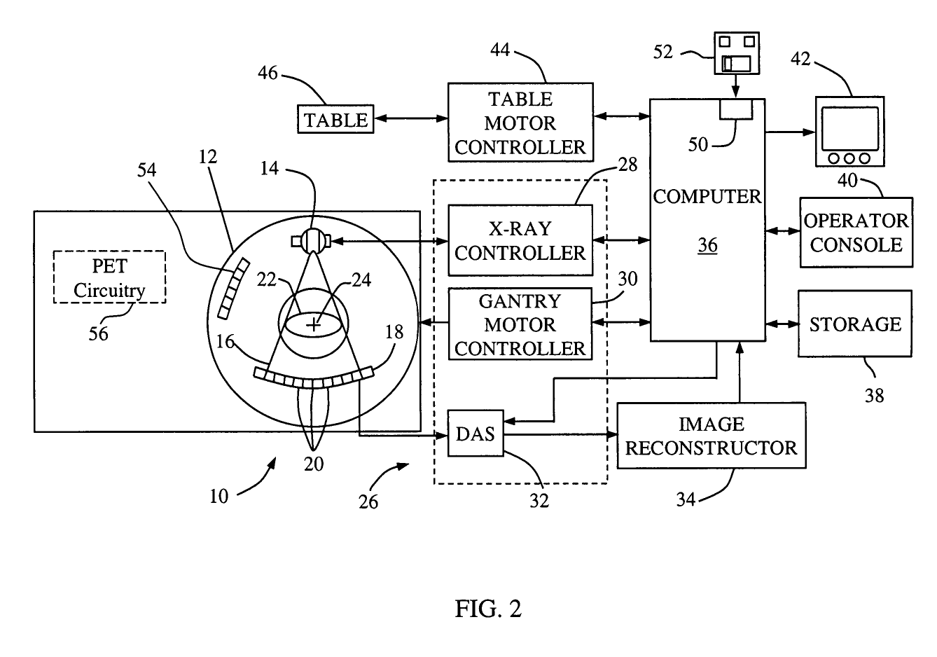

Referring to FIGS. 1 and 2, a multi-slice scanning imaging system, for example, a CT imaging system 10, is shown as including a gantry 12 representative of a “third generation” CT imaging system. Gantry 12 has an X-ray source 14 that projects a beam of X-rays 16 toward a detector array 18 on the opposite side of gantry 12. Detector array 18 is formed by a plurality of detector rows (not shown) including a plurality of detector elements 20 which together sense the projected X-rays that pass through an object, such as a medical patient 22. Each detector element 20 produces an electrical signal that represents the intensity of an impinging X-ray beam and hence allows estimation of the attenuation of the beam as it passes through object or patient 22. During a scan to acquire X-ray projection data, gantry 12 and the components mounted thereon rotate about a center of rotation 24.

FIG. 2 shows only a detector row of detector elements 20. However, multislice detector array 18 includes a plurality of parallel detector rows of detector elements 20 such that projection data corresponding to a plurality of quasi-parallel or parallel slices can be acquired simultaneously during a scan.

Rotation of gantry 12 and the operation of X-ray source 14 are governed by a control mechanism 26 of CT system 10. Control mechanism 26 includes an X-ray controller 28 that provides power and timing signals to X-ray source 14 and a gantry motor controller 30 that controls the rotational speed and position of gantry 12. A data acquisition system (DAS) 32 in control mechanism 26 samples analog data from detector elements 20 and converts the data to digital signals for subsequent processing. An image reconstructor 34 receives sampled and digitized X-ray data from DAS 32 and performs high-speed image reconstruction. The reconstructed image is applied as an input to a computer 36 which stores the image in a storage device 38.

Computer 36 also receives commands and scanning parameters from an operator via console 40 that has a keyboard. An associated cathode ray tube display 42 allows the operator to observe the reconstructed image and other data from computer 36. The operator supplied commands and parameters are used by computer 36 to provide control signals and information to DAS 32, X-ray controller 28 and gantry motor controller 30. In addition, computer 36 operates a table motor controller 44 which controls a motorized table 46 to position patient 22 in gantry 12. Particularly, table 46 moves portions of patient 22 through gantry opening 48.

In one embodiment, computer 36 includes a device 50, for example, a floppy disk drive or CD-ROM drive, for reading instructions and/or data from a computer-readable medium 52, such as a floppy disk or CD-ROM. In another embodiment, computer 36 executes instructions stored in firmware (not shown). Computer 36 is programmed to perform functions described herein, and as used herein, the term computer is not limited to just those integrated circuits referred to in the art as computers, but broadly refers to computers, processors, microcontrollers, microcomputers, programmable logic controllers, application specific integrated circuits, and other programmable circuits, and these terms are used interchangeably herein.

Although the specific embodiment mentioned above refers to a third generation CT system, methods for analyzing an abnormality of an object equally apply to fourth generation CT systems that have a stationary detector and a rotating X-ray source, fifth generation CT systems that have a stationary detector and an X-ray source.

Additionally, although the herein described methods are described in a medical setting, it is contemplated that the benefits of the methods accrue to non-medical imaging systems such as those systems typically employed in an industrial setting or a transportation setting, such as, for example, but not limited to, a baggage scanning system for an airport, other transportation centers, government buildings, office buildings, and the like. The benefits also accrue to micro PET and CT systems which are sized to study lab animals as opposed to humans.

It is noted that CT imaging system 10 can be combined with a PET imaging system, that is described below, to form a PET-CT imaging system (not shown). In one embodiment, the PET-CT imaging system includes a plurality of PET detectors 54, rotating rod sources (not shown) and a PET circuitry 56 within gantry 12. An example of such as PET-CT system is a Discovery LS PET-CT system commercially available from General Electric Medical Systems, Waukesha, Wis. In another embodiment, the PET-CT imaging system includes the plurality of PET detectors 54 and PET circuitry 56 located with a separate gantry. An example of such a PET-CT system is a Discovery ST system commercially available from General Electric Medical Systems.

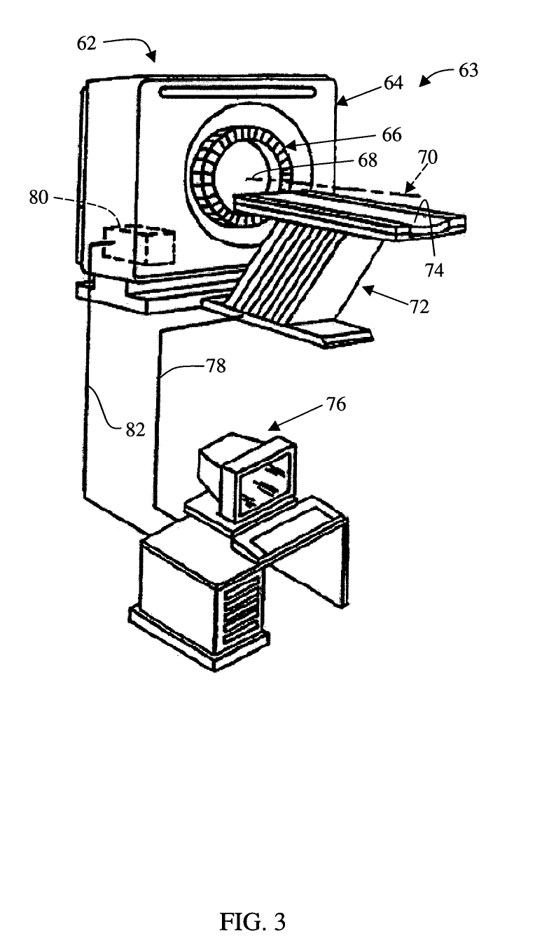

FIG. 3 is an isometric view of an embodiment of a PET imaging system 62 in which methods for combining an anatomic structure and metabolic activity for an object are implemented. PET imaging system 62 includes a PET scanner 63. PET scanner 63 includes a gantry 64 which supports a detector ring assembly 66 about a central opening, or bore 68. Detector ring assembly 66 is circular in shape, and is made up of multiple detector rings (not shown) that are spaced along a central axis 70 to form a cylindrical detector ring assembly. A table 72 is positioned in front of gantry 66 and is aligned with central axis 70 of detector ring assembly. A table controller (not shown) moves a table bed 74 into bore 68 in response to commands received from an operator work station 76 through a serial communications link 78. A gantry controller 80 is mounted within gantry 64 and is responsive to commands received from operator work station 76 through a second serial communication link 82 to operate gantry 64.

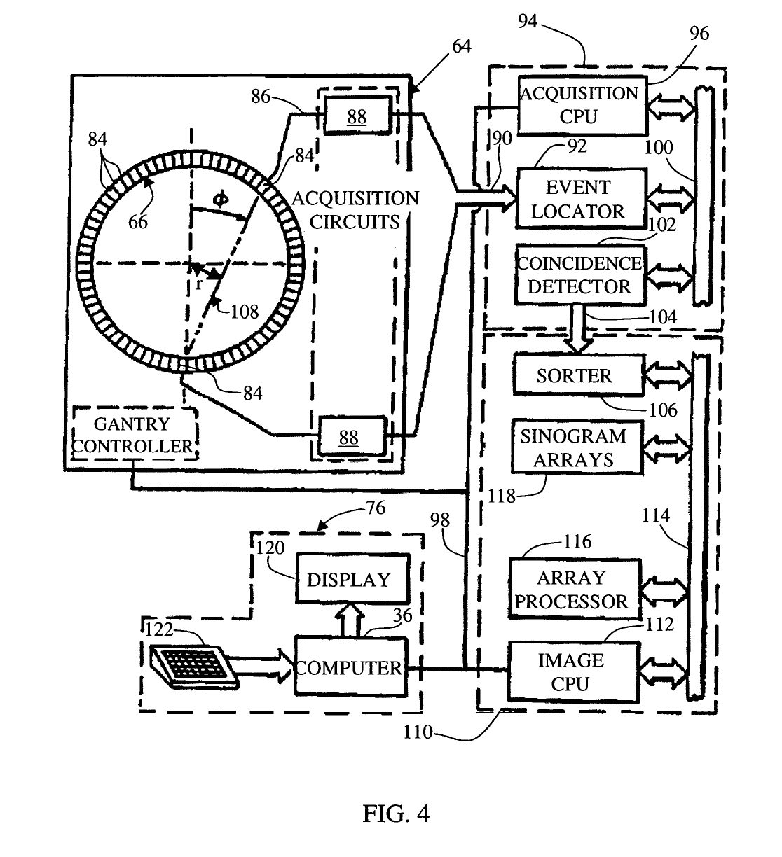

FIG. 4 shows a block diagram of PET imaging system 62 of FIG. 3. Each detector ring of detector ring assembly 66 includes detectors 84. Each detector 84 includes scintillator crystals (not shown). Each scintillator crystal is disposed in front of a photomultiplier tube (PMT) (not shown). PMTs produce analog signals on line 86 when a scintillation event occurs at one of the scintillator crystals that are disposed in front of the PMTs. The scintillation event occurs when a photon is received by one of the scintillator crystals. In one embodiment, photons are generated by administering a compound, such as, ¹¹C-labeled glucose, ¹⁸F-labeled glucose, ¹³N-labeled ammonia and ¹⁵O-labeled water within the object, an emission of positrons by the compounds, a collision of the positrons with free electrons of the object, and generation of simultaneous pairs of photons. Alternatively, the photons are transmitted by rotating rod sources within a FOV of PET imaging system 62. A set of acquisition circuits 88 is mounted within gantry 64 to receive the signals and produce digital signals indicating event coordinates (x,y) and total energy. These are sent through a cable 90 to an event locator circuit 92 housed in a separate cabinet. Each acquisition circuit 88 also produces an event detection pulse (EDP) which indicates the exact moment the scintillation event took place.

Event locator circuits 92 form part of a data acquisition processor 94 which periodically samples the signals produced by acquisition circuits 88. Processor 94 has an acquisition central processing unit (CPU) 96 which controls communications on a local area network 98 and a backplane bus 100. Event locator circuits 92 assemble the information regarding each valid event into a set of digital numbers that indicate precisely when the event took place and the position of a scintillation crystal which detected the event. This event data packet is conveyed to a coincidence detector 102 which is also part of data acquisition processor 94. Coincidence detector 102 accepts the event data packets from event locators 92 and determines if any two of them are in coincidence. Events which cannot be paired are discarded, but coincident event pairs are located and recorded as a coincidence data packet that is conveyed through a serial link 104 to a sorter 106.

Each pair of event data packets that is identified by coincidence detector 102 is described in a projection plane format using four variables r, v, θ, and Φ. Variables r and Φ identify a plane 108 that is parallel to central axis 70, with Φ specifying the angular direction of the plane with respect to a reference plane and r specifying the distance of the central axis from the plane as measured perpendicular to the plane. Variables v and θ (not shown) further identify a particular line within plane 108, with θ specifying the angular direction of the line within the plane, relative to a reference line within the plane, and v specifying the distance of center from the line as measured perpendicular to the line.

Sorter 106 forms part of an image reconstruction processor 110. Sorter 106 counts all events occurring along each projection ray, and stores that information in the projection plane format. Image reconstruction processor 110 also includes an image CPU 112 that controls a backplane bus 114 and links it to local area network 98. An array processor 116 also connects to backplane bus 114. Array processor 116 converts the event information stored by sorter 106 into a two dimensional sinogram array 118. Array processor 116 converts data, such as, for instance, emission data that is obtained by emission of positrons by the compound or transmission data that is obtained by transmission of photons by the rotating rod sources, from the projection plane format into the two-dimensional (2D) sinogram format. Examples of the 2D sinogram include a PET emission sinogram that is produced from emission data and a PET transmission sinogram that is produced from transmission data. Upon conversion of the data into the two-dimensional sinogram format, images can be constructed. Operator work station 76 includes computer 36, a cathode ray tube (CRT) display 120, and a keyboard 122. Computer 36 connects to local area network 98 and scans keyboard 122 for input information. Through keyboard 122 and associated control panel switches, the operator controls calibration of PET imaging system 62, its configuration, and positioning of table 72 for a PET scan. Similarly, once computer 36 receives a PET image and a CT image, the operator controls display of the images on CRT display 120. On receipt of the PET image and the CT image, computer 36 perform s a method for combining an anatomic structure and metabolic activity for an object, such as patient 22.

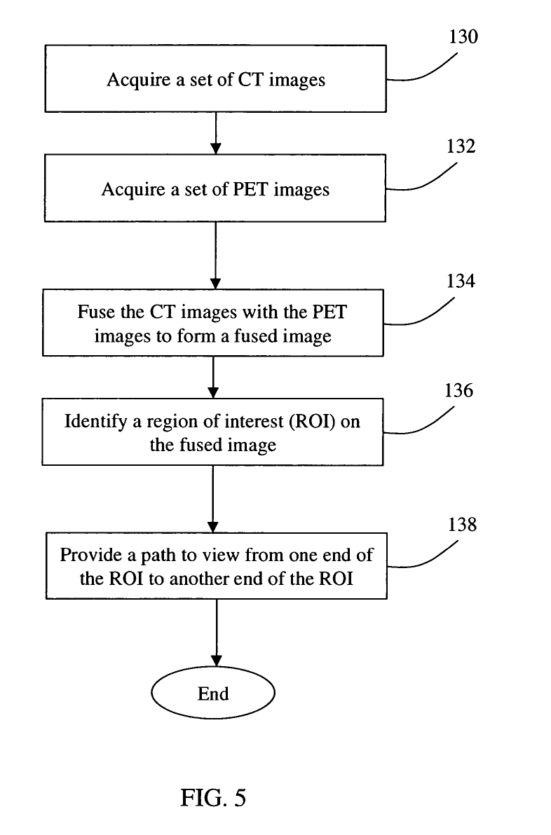

FIG. 5,is a flowchart of an embodiment of the invention for combining an anatomic structure and metabolic activity for an object, such as patient 22. The method is executed by computer 36. The method is stored in storage 38 or computer-readable medium 52. The method includes acquiring (at step 130) a set of CT images. The CT images are acquired from image reconstructor 34. The CT images are generated by scanning patient 22 using CT system 10. The method further includes acquiring (at step 132) a set of PET images. The PET images are acquired from image CPU 112. The PET images are generated by scanning patient 22 using PET system 62. In an alternative embodiment, the PET images and the CT images are acquired by using the PET-CT system that is described above.

The method further includes fusing (at step 134) the CT images and the PET images to form a 3-dimensional (3D) fused image. In one embodiment, the fused image is a fused image of a colon cavity of patient 22. In another embodiment, the fused image is a fused image of an inside wall of the colon of patient 22. In yet another embodiment, the fused image is an image of an outside wall of the colon of patient 22.

The CT and the PET images can be fused at step 134 by statistical methods or color-wash methods. The color-wash methods assign a color scale to one image, such as a PET image, and an intensity scale to the other image, such as a CT image. For instance, the PET image is assigned a color scale ranging from violet to red colors and each CT image has an intensity scale ranging from a high intensity to a low intensity. Statistical methods select the most significant values from each of the CT and PET images and assign as many orthogonal colors to each as possible for display by display device 42. For example, a pixel with a most significant bit on the CT image is assigned a blue color and a pixel with a most significant value on the PET image is assigned a red color. A pixel with a next most significant bit on the CT image is assigned a lighter blue color. A pixel with a next most significant bit on the PET image is assigned a lighter red color. The remaining pixels on the CT and the PET images are assigned even lighter blue and red colors, respectively.

The method continues by identifying (at step 136) a region of interest (ROI) on the fused image. The ROI corresponds to an organ of interest of patient 22. Examples of organs of interest include a colon of patient 22 and bronchial tubes of the patient. The ROI is identified by distinguishing a density of the ROI from the densities of voxels of regions outside the ROI. As an example, the difference in the density of the ROI from the densities of regions outside the ROI is created by inflating the organ of interest with air or gas, such as, carbon dioxide. As another example, the difference in the density of the ROI from the densities of regions outside the ROI is created by. Densities are extracted from volume arrays of the ROI and of regions outside the ROI by trilinear interpolation. For example, a volume V_{xyz} of a voxel within the ROT and located at a position (x,y,z) is calculated by
V_{xyz}=V₀₀₀(1−x)(1−y)(1−z)+V₁₀₀x(1−y)(1−z)+V₀₁₀(1−x)y(1−z)+V₀₀₁(1−x)(1−y)z+V₁₀₁x(1−y)z+V₀₁₁x(1−x)yz+V₁₁₀xy(1−z)+V₁₁₁xyz, (1)
where V₀₀₀ is a volume of a voxel that is located at a vertex (0,0,0) of a cube encompassing the voxel with the volume V_{xyz},V₁₀₀ is a volume of a voxel that is located at a vertex (1,0,0) of a cube encompassing the voxel with the volume V_{xyz}, V₀₁₀, is a volume of a voxel that is located at a vertex (0,1,0) of the cube encompassing the voxel with the volume V_{xyz}, V₀₀₁ is a volume of a voxel that is located at a vertex (0,0,1) of the cube encompassing the voxel with the volume V_{xyz}, V₁₀₁ is a volume of a voxel that is located at a vertex (1,0,1) of the cube encompassing the voxel with the volume V_{xyz}, V₀₁₁ is a volume of a voxel that is located at a vertex (0,1,1) of the cube encompassing the voxel with the volume V_{xyz}, V₁₁₀ is a volume of a voxel that is located at a vertex (1,1,0) of the cube encompassing the voxel with the volume V_{xyz}, V₁₁₁ is a volume of a voxel that is located at a vertex (1,1,1) of the cube encompassing the voxel with the volume V_{xyz}, Density of the voxel with volume V_{xyz} is obtained from a weight of the voxel and the volume of the voxel.

The method further includes defining (at step 138) a path to view from one end of the ROI to another end of the ROI. The path is calculated by applying a special case of Green's theorem surface to calculate centroids along estimated paths along the ROI and by connecting the centroids. The special case of Green's theorem is provided by:
∫∫∫(u∇²v−v∇²u)dV=∫∫N(u∇v−v∇u)dS (2)
where N is a normal to the surface S which bounds volume V surrounding the ROI, and u(x,y,z) and v(x,y,z) are scalar fields within the volume V and have continuous second partial derivatives. In one embodiment, the path is provided from one point on an axial line passing through a center of the ROI to another point located on the axial line. For example, the path may be provided from an anal verge of patient 22 to cecum of patient 22. As another example, the path may be provided from the cecum to the anal verge. A user, such as a physician, can then traverse the path and view the inside of the colon that the physician is able to view with an image obtained by performing a colonoscopy but without making patient 22 undergo any inconveniences of the colonoscopy.

In an alternative embodiment, the method includes determining whether the organ of interest of patient 22 is inflated with at least one of gas and air to create a difference in density of the ROI from densities of regions of patient 22 outside the ROI. The method includes executing steps 130, 132, 134, 136, and 138 if it is determined that the organ of interest is inflated. Optionally, the method may not execute steps 130, 132, 134, 136, and 138 if it is determined that the organ of interest is not inflated.

Yet another alternative embodiment of the method includes determining whether patient 22 has been prepared for a computed tomograph colonography. An example of the preparation for the computed tomograph colonograpy includes inflating a colon of patient 22 with carbon dioxide. If it is determined that patient 22 has been prepared, the method further includes determining whether the computed tomograph colonography has been performed on patient 22. If it is determined that the computed tomograph colonography has been performed, the method continues by determining whether a PET scan has been performed on patient 22. The method further includes executing steps 130, 132, 134, 136, and 138 on determining that the PET scan has been performed.

Still another alternative embodiment of-the method includes determining whether patient 22 has not been prepared for a computed tomograph colonography and/or a colonoscopy. For example, instead of clearing a colon of patient 22, a PET exam would help differentiate between fecal matter and malignant polyps. The method further includes determining whether the computed tomography colonography has been performed on determining that the patient 22 has not been prepared. The method continues by determining whether a PET scan has been performed on determining that the computed tomograph colonography has been performed. The method further includes executing steps 130, 132, 134, 136, and 138 on determining that the PET scan has been performed.

Another embodiment of the method includes determining whether at least one of supine and prone CT acquisitions have not been performed when performing a CT colonography on patient 22. The method continues by determining whether a PET scan has been performed on determining that at least one of supine and prone CT acquisitions have not been performed. The method further includes executing steps 62, 64, 66, 68, and 70 on determining that the PET scan has been performed.

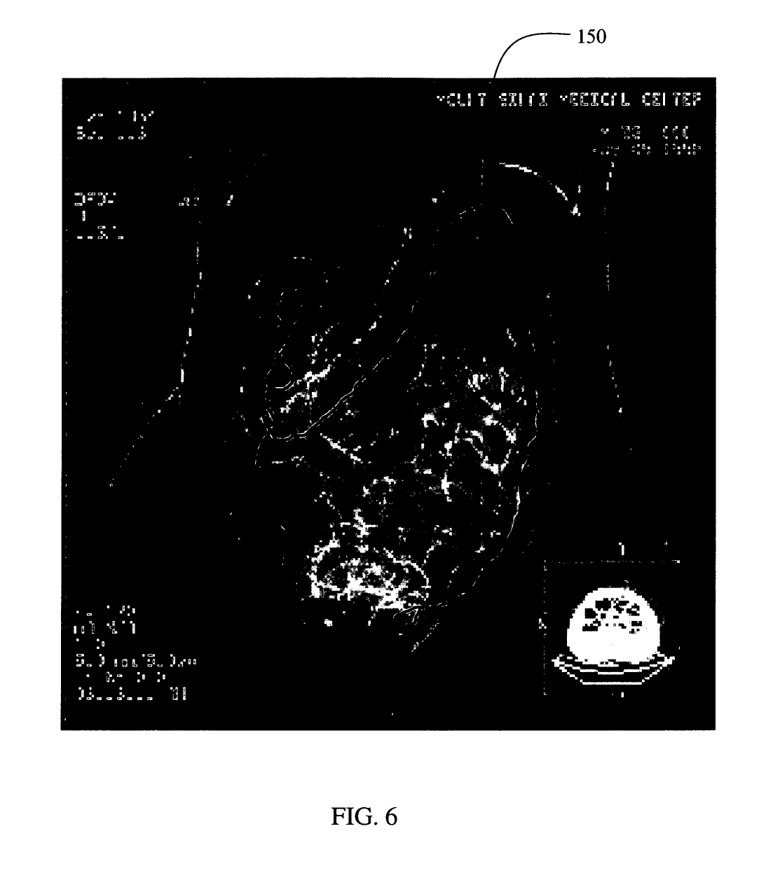

FIG. 6 shows an image of a colon of patient 22 to illustrate a method for combining an anatomical structure and metabolic activity acquired from patient 22 during a medical examination. The image in FIG. 6 shows a path 150, as a black solid line, along which a user, such as a physician, of the PET-CT system views the ROI within the colon cavity. The fused PET and CT data from a fused 3-dimensional (3D) volume that includes the colon. The user is afforded the ability with a display to progressively advance from one end of the colon cavity to another end of the colon cavity. The user can start viewing at any point on the path and can end viewing at any point on path 150. In an alternative embodiment, path 150 is not centered axially within the colon cavity but is displaced from the a center axis of the colon. An axial 2D CT image of the colon cavity is also shown at the bottom right corner of the large image of the colon. In one embodiment, display 42 simultaneously displays a fused image of the colon cavity and the axial 2D CT image of the colon cavity from a viewpoint. The simultaneous display aids the user in diagnosis of polyps inside the colon cavity. In an alternative embodiment, display 42 displays the fused image of an outside wall of the colon and simultaneously displays a sagital 2D CT image of the outside wall from a viewpoint. In yet another embodiment, display 42 displays the fused image of an inside wall of the colon and simultaneously displays a coronal 2D CT image of the inside wall from a viewpoint.

Hence, the herein described systems and methods provide fused images and/or a path to view the fused images through a fused volume. The fused volume and the path along which the fused images are viewed provide the viewing results of a CT colonoscopy but without patient 22 undergoing inconveniences that patient undergoes when preparing for the CT colonoscopy. In addition, patient 22 does not feel intruded as patient 22 feels during the CT colonosocopy when a camera intrudes into the colon of patient 22. The systems and methods aids the user in detecting polyps that could potentially be cancerous. The fused volume also enables the user to analyze anatomic structures and metabolic activity that is not viewable in a conventional CT colonoscopy. For instance, the colon wall and the area immediately outside the colon wall may be viewed.

It is to be noted that in an alternative embodiment, a single photon emission computed tomography (SPECT) imaging system, instead of PET system 62, can be used to obtain SPECT images of the object.

While the invention has been described in terms of various specific embodiments, those skilled in the art will recognize that the invention can be practiced with modification within the spirit and scope of the claims.

1. A method for combining an anatomic structure and metabolic activity for an object, the method comprising:
acquiring a first set of images by scanning the object using a first modality;
acquiring a second set of images by scanning the object using a second modality;
fusing the first and second sets of images to form a fused volume;
identifying a region of interest (ROT) in the fused volume, the ROT corresponding to an organ of interest of the object; and
providing a viewing path through the fused volume at least partially following the ROI.

2. A method in accordance with claim 1 further comprising:
inflating the organ of interest with at least one of gas and air to create a difference in density of the ROI from densities of regions outside the ROI.

3. A method in accordance with claim 2 wherein identifying the ROI comprises identifying the ROI by distinguishing the density of the ROI from the densities of regions outside the ROI.

4. A method in accordance with claim 1 wherein providing the viewing path comprises providing the ROI that may be viewed in both directions along the viewing path.

5. A method in accordance with claim 1 further comprising preparing the object for a computed tomograph colonography.

6. A method in accordance with claim 1 further comprising foregoing preparation of the object for a computed tomograph colonography.

7. A method in accordance with claim 1 further comprising foregoing at least one of supine and prone computed tomography (CT) acquisitions.

8. A method in accordance with claim 1 further comprising:
displaying the fused image; and
displaying at least one of an axial 2-dimensional (2D) CT image of the organ of interest, a sagital 2D CT image of the organ of interest, and a coronal 2D CT image of the organ of interest.

9. A computer-readable medium encoded with a program configured to instruct a computer to:
fuse at least two of computed tomography (CT) data, single photon emission computed tomography (SPECT) data, and positron emitted tomography (PET) images to form a fused data set;
identify a region of interest (ROI) in the fused data set, the ROI corresponding to an organ of interest of an object; and
provide a path through the fused data set along which to view the fused data set.

10. A computer-readable medium in accordance with claim 9 wherein the program is configured to:
determine whether the organ of interest is inflated with at least one of gas and air to create a difference in density of the ROI from densities of regions outside the ROI; and
execute if the organ of interest has been inflated.

11. A computer-readable medium in accordance with claim 9 wherein to identify the ROI the computer program configured to distinguish the density of the ROI from the densities of regions outside the ROI.

12. A computer-readable medium in accordance with claim 9 wherein to provide the path the computer program configured to provide a path from one point on an axial line passing through a center of the ROI to another point located on the axial line.

13. A computer-readable medium in accordance with claim 9 wherein the computer program is configured to:
determine whether the object has been prepared for a computed tomograph colonography; and
determine whether the computed tomography colonography has been performed on determining that the object has been prepared.

14. A computer-readable medium in accordance with claim 9 wherein the computer program is configured to:
determine whether the object has been prepared for a computed tomograph colonography; and
determine whether the computed tomography colonography has been performed on determining that the object has been prepared;
determine whether a PET scan has been performed on determining that the computed tomograph colonography has been performed; and
execute if the PET scan has been performed.

15. A computer-readable medium in accordance with claim 9 wherein at least two of the CT data, the PET data, and the CT data, the computer program configured to fuse at least two of the CT data, the PET data, and the CT data to obtain a fused volume of a colon cavity, an inside wall of the colon, and an outside wall of the colon.

16. A computer-readable medium in accordance with claim 9 wherein the computer program is configured to:
check whether a preparation of the object for a computed tomograph colonography has not been performed; and
execute if the preparation has not been performed.

17. A computer-readable medium in accordance with claim 9 wherein the computer program is configured to:
check whether at least one of supine and prone CT acquisitions has been foregone; and
execute if at least one of the supine and prone CT acquisitions has been foregone.

18. A computer-readable medium in accordance with claim 9 wherein the computer program is configured to:
instruct a, display device to display a fused image corresponding to the fused data set; and
instruct the display device to display at least one of an axial 2-dimensional (2D) CT image of the organ of interest, a sagital 2D CT image of the organ of interest, and a coronal 2D CT image of the organ of interest.

19. A computer programmed to:
fuse computed tomography (CT) images and positron emission tomography (PET) images to form a fused volume;
identify a region of interest (ROI) in the fused volume, the ROI corresponding to an organ of interest of the object; and
provide a viewing path through the fused volume at least partially following the ROI.

20. An imaging system for combining an anatomic structure and metabolic activity for an object, the imaging system comprising:
a radiation source;
a radiation detector; and
a controller operationally coupled to the radiation source and the radiation detector, the controller configured to:
acquire computed tomography (CT) images generated by performing a CT colonography;
acquire positron emission tomography (PET) images generated by performing a PET scan of a colon of the object;
fuse the CT images and PET images to form a fused volume;
identify a region of interest (ROI) in the fused volume, the ROI corresponding to the colon; and
provide a viewing path through the fused volume of interest partially following the ROI.

21. An imaging system in accordance with claim 20 wherein the controller is further configured to:
determine whether the colon is inflated with at least one of gas and air to create a difference in density of the ROI from densities of regions outside the ROI; and
execute if the colon has been inflated.

22. An imaging system in accordance with claim 20 wherein to provide the viewing path the controller configured to provide the ROI that may viewed in both directions.

23. An imaging system for combining an anatomic structure and metabolic activity for an object, the imaging system comprising:
a radiation source;
a radiation detector; and
a controller operationally coupled to the radiation source and the radiation detector, the controller configured to:
acquire computed tomography (CT) images generated by scanning the object using a first modality;
acquire positron emission tomography (PET) images generated by scanning the object using a second modality;
fuse the CT images and PET images to form a fused volume;
identify a region of interest (ROI) in the fused volume, the ROI corresponding to an organ of interest of the object; and
provide a viewing path through the fused volume at least partially following the ROI.

24. An imaging system in accordance with claim 23 wherein the controller is further configured to:
determine whether the organ of interest is inflated with at least one of gas and air to create a difference in density of the ROI from densities of regions outside the ROI; and
execute if the organ of interest has been inflated.

25. An imaging system in accordance with claim 23 wherein to provide the viewing path the controller configured to provide the ROI that may be viewed in both directions along the ROI.
